(19) United States
(12) Patent Application Publication
(10) Pub. No.: US 2014/0290792 A1
(43) Pub. Date: Oct. 2, 2014

(54) Connector for a Drug Delivery Device Reservoir

(71) Applicants: Richard James Vincent Avery, Chipping Campden (GB); Joseph Butler, Warwickshire (GB)

(72) Inventors: Richard James Vincent Avery, Chipping Campden (GB); Joseph Butler, Warwickshire (GB)

(73) Assignee: SANOFI-AVENTIS DEUTSCHLAND GMBH, Frankfurt am Main (DE)

(21) Appl. No.: 13/814,949

(22) PCT Filed: Aug. 11, 2011
(86) PCT No.: PCT/EP11/63840

§ 371 Date: Apr. 22, 2013

(30) Foreign Application Priority Data
Aug. 13, 2010 (US) 61373383
Oct. 26, 2010 (EP) 10188848.5

(51) Int. Cl. A61J 1/20; A61M 5/31

(52) U.S. Cl. CPC A61J 1/2096; A61M 5/31; A61M 2005/3142; A61J 2001/2048
USPC 141/18

(57) ABSTRACT

A connector for use with a reservoir. The connector comprises a main body defining a bore extending from a proximal end to a distal end of the main body. The bore is configured for receiving the reservoir. An inner wall extends from the proximal end to the distal end. At least a portion of the inner wall is configured for attachment to an outer surface of the reservoir. An outer wall extends from the proximal end and the distal end. The outer wall is configured for attachment to a drug delivery device. In one arrangement, the outer wall is configured for attachment to a cartridge holder of the drug delivery device.

### CROSS REFERENCE TO RELATED APPLICATIONS

The present application is a U.S. National Phase Application pursuant to 35 U.S.C. §371 of International Application No. PCT/EP2011/063840 filed Aug. 11, 2011, which claims priority to U.S. Patent Application No. 61/373,383 filed Aug. 13, 2010 and European Patent Application No. 10188848.5 filed Oct. 26, 2010. The entire disclosure contents of these applications are herewith incorporated by reference into the present application.

### FIELD OF INVENTION

The present patent application is generally directed to reservoirs, particularly reservoirs containing a medicament. More particularly, the present application is generally directed to a connector for use with a reservoir and a reservoir holder, e.g. so as to prevent unwanted reservoir cross use. As just one example, such medicament reservoirs may comprise an ampoule, a cartridge, a vial, or a pouch, and may be used with a medical delivery device. Exemplary medical or drug delivery devices include, but are not limited to syringes, pen type injection syringes, injectors, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen type injection syringe or infused via a pump. With respect to certain known reusable pen type drug delivery devices, a patient loads a cartridge containing the insulin into a proximal end of a cartridge holder. After the cartridge has been correctly loaded, the user may then be called upon to select a dose of medicament or medicaments contained within the cartridge. Multiple doses may be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a disposable device, once the cartridge no longer contains any medicament, the user is recommended to dispose of the entire device.

Such known self administration systems requiring the removal and reloading of spent cartridges have certain limitations. For example, in certain generally known systems, a user simply loads a new cartridge into the delivery system without the drug delivery device or without the cartridge having any mechanism of preventing cross use of an incorrect cartridge. That is, the drug delivery device does not have a mechanism for determining or is not configured to determine if the medicament contained in the cartridge is indeed the correct type of medicament to be administered by the patient.

Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short acting insulin in lieu of a long insulin could result in injury or even death.

Some drug delivery devices or systems may use a color coding scheme to assist a user and/or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that such disposable cartridges are manufactured in essentially standard sizes and manufactured to comply with certain recognized local and international standards. Consequently, such cartridges are typically supplied in standard sized cartridges (e.g., 3 ml cartridges). Therefore, there may be a variety of cartridges supplied by a number of different suppliers and containing a different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge.

### SUMMARY

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of a medicament or an incorrect titration of such a medicament) from such a drug delivery device.

There is, therefore, a general need to physically dedicate or mechanically code a cartridge to its drug type. There is also a general need to design an injection device that only accepts or functions with the dedicated or coded features provided on and/or with the cartridge so as to prevent unwanted cartridge cross use. Similarly, there is also a general need for a dedicated cartridge that allows the drug delivery device to be used with only an authorized cartridge containing a specific medicament while also preventing undesired cartridge cross use.

There is also a general need to provide a dedicated cartridge that is difficult to tamper with so that the cartridge may not be compromised in that the cartridge can be used with an unauthorized drug or drug delivery device. Because such cartridges may be difficult to tamper with, such cartridges may also help to reduce the risk of counterfeiting: i.e., making it more difficult for counterfeiters to provide unregulated counterfeit medicament carrying products.

Coding such cartridges to a specific cartridge holder would also provided certain manufacturing and assembly benefits such as in the manufacturing of a disposable (non-reusable) pen type drug delivery device. For example, coding a specific cartridge to a specific cartridge housing or holder (and no other cartridge housing or holder) could provide a certain degree of manufacturing certainty that the correct cartridge and therefore the desired medicament has been indeed loaded into a correct cartridge holder.

It is an object of the present disclosure to provide one or more means which facilitate provision of a novel drug delivery device or novel elements for a drug delivery device. In particular, it is intended to provide one or more means which facilitate overcoming one, more or all of the above-mentioned problems.

This object is achieved, for example, by the subject matter of the independent claim. Advantageous refinements are the subject matter of the dependent claims. As it is readily apparent from the disclosure below, various ways to provide for novel elements for drug delivery devices are disclosed herein. Accordingly, the disclosure covers solutions which are different from the ones currently claimed.

One aspect of this disclosure relates to a connector. The connector may be a connector for use with a reservoir, e.g. comprising a cartridge. The connector may comprise a body, particularly a unitary body. The body may be configured for attachment to the reservoir. The connector may be attached to the reservoir via the body. The connector may be adapted to be attached to a proximal portion or a proximal end of the reservoir. The connector may be adapted to cooperate with or to attach to a drug delivery device. In particular, the connector may be adapted to cooperate with or to attach to a receiving member of the drug delivery device. The receiving member may be or may comprise a reservoir holder, particularly a cartridge holder.

The connector may be adapted to interact with one or more elements of a drug delivery device. By means of differently embodied connectors which may be used with standard reservoirs, the standard reservoirs can be easily adapted to be used with different devices.

One aspect relates to an assembly or a system comprising a reservoir and a connector, preferably a connector as described previously. The connector may be attached to the reservoir. In particular, the connector may be attached to an outer surface of the reservoir, preferably via the body. The connector, in particular an outer wall thereof may be configured to cooperate with or to attach to a receiving member of a drug delivery device. The connector may be firmly secured to the reservoir, in particular such that its position with respect to the reservoir is stable. The connector may be attached to the reservoir by a frictional connection.

A further aspect relates to an assembly. The assembly may comprise a reservoir. The reservoir may be a cartridge. The assembly may further comprise a connector. He connector may comprise the previously describe connector. The connector may be for use with the reservoir. Said connector may comprise a body. The body may be configured for attachment to the reservoir. The connector may be adapted to be attached to a proximal portion of the reservoir. A heat shrink label may be positioned over a portion of said reservoir and said connector. The heat shrink label may be positioned so that said heat shrink label maintains said connector on said reservoir. Said heat shrink label may terminate on or in a groove provided by said connector.

One aspect relates to a drug delivery device. The device may comprise a reservoir and a connector, which is expediently attached to the reservoir. The connector cooperates with or is attached to a reservoir holder of the drug delivery device. Expediently, the drug delivery device comprises an assembly or a system as described further above.

Features which are described herein in connection with the connector, the assembly and the drug delivery device may also be used in conjunction with an element in connection with which they are not explicitly described, e.g. in connection with the system, the assembly, the connector or the drug delivery device as the case may be.

In one embodiment, the connector, in particular the body, comprises a main body. The connector may be formed unitarily. The main body may define a bore or opening extending from a proximal end to a distal end of the main body. The bore or opening may be configured for receiving the reservoir. An inner wall, in particular an inner wall of the bore or opening, may extend from the proximal end to the distal end. At least a portion of the inner wall may be configured for attachment to an outer surface of the reservoir. An outer wall, in particular an outer wall of the body, may extend from the proximal end and the distal end. The outer wall may be configured for attachment to or cooperation with the drug delivery device.

In one embodiment, the connector is a multi-component connector. The connector, in particular the body, may comprise an inner body. The inner body may be configured for attachment, preferably frictional attachment, to an outer surface of the reservoir. The connector, in particular the body, may comprise an outer body. The outer body, in particular an inner surface of the outer body, may have a connection mechanism or a connection feature for connecting to the inner body. The outer and the inner body may be connected when the connector is attached to the reservoir.

In one embodiment, the inner body comprises a plurality of fingers. The fingers may be inwardly directed. The fingers may be radially directed. The fingers may be inwardly directed radially fingers. The fingers are expediently arranged to provide or the fingers provide frictional engagement with the outer surface of the reservoir, in particular for the frictional attachment of the inner body to the outer surface of the reservoir. When the outer body is connected to the inner body, the fingers may be moved or may be pressed radially inwardly. In this way the frictional connection of the inner body to the reservoir may be enhanced.

In one embodiment, the connection mechanism of the outer body comprises a thread. The thread may be used to connect to a thread provided on an external surface of the inner body. Alternatively or additionally, the connection mechanism of the outer body comprises a rib. The rib may be used to connect to a groove provided on an external surface of the inner body.

In one embodiment, the connector comprises a flexible connector. The connector may, for example, be flexible insofar as the connector may be configured to adapt the shape of the inner wall of the body to the reservoir. The connector or the inner wall may be, in particular permanently or temporarily, deformable. The connector or the inner wall may be elastically or plastically deformable. A deformable connector or inner wall facilitates adapting the shape to the one of the reservoir. The deformation may take place when or before the connector is attached to the reservoir.

In one embodiment, the flexible connector comprises a flexible feature. The flexible feature ma ybe provided to flexibly vary an extension of the connector, such as its circumference. Flexible features are particular suitable for a one-piece or unitary connector. Alternatively or additionally, the flexible connector may comprise a multi-component flexible connector, which may comprise an inner body and an outer body as described further above, for example. The fingers of the inner body may be flexible, for example. Alternatively or additionally, the flexible connector may comprise a plurality of crush ribs. The crush ribs may be, particularly permanently, deformed when the connector is attached to the reservoir.

In one embodiment, the connector comprises a heat shrink material. A connector of this kind may be shrunk on the reservoir to firmly attach the connector to the reservoir.

In one embodiment, the connector is positionable or configured to be positionable along an external surface of the reservoir by sliding the connector onto a proximal end of the reservoir. Expediently, the connector is positionable in this way before it is firmly secured to the reservoir.

In one embodiment, the connector comprises a snap fit connector. In this way, the connector may snap together, particularly around a portion of the reservoir. Two free ends of the connector in the unassembled state may be connected with each other when the connector is assembled with the reservoir.

In one embodiment, the assembly or system comprises a heat shrink member, such as a heat shrink label. The heat shrink member may be positioned over a portion of the reservoir and a portion of the connector, in particular such that the heat shrink member maintains the connector on the reservoir.

In one embodiment, the connector, in particular the outer surface of the connector, comprises a groove. The groove may extend azimuthally along the outer surface.

In one embodiment, the heat shrink member terminates on or in the groove provided by the connector.

In one embodiment, the reservoir comprises a proximal end and a distal end.

In one embodiment, the connector is positioned or adapted to be positioned near or at the proximal end of the reservoir.

The term “proximal end” as used herein in conjunction with the connector, the assembly, the system or the drug delivery device or an element thereof may be that end which is or is to be arranged furthest away from a distal end of the drug delivery device or any of the recited elements. The “distal end” of the drug delivery device or any of the elements recited previously may be that end which is or is to be arranged closest to the dispensing end of the device or the distal end of an element of the device.

The distal end of the reservoir may be that end, where the reservoir has an opening or port. The opening or port may be covered by a septum to retain drug within the reservoir. The septum may be pierceable by a needle. The proximal end of the reservoir may be the end which is furthest away from the distal end.

In one embodiment, the connector is positioned at a proximal portion of the reservoir.

The connector may be arranged closer at the proximal end than at the distal end of the reservoir.

In one embodiment, the connector comprises a retention feature. The retention feature may be provided on the body of the connector. The retention feature may engage a groove provided along a proximal portion of the reservoir.

In one embodiment, the reservoir comprises a cartridge. The cartridge may comprise a tubular member. The tubular member may comprise a bung located near a proximal end of the tubular member. The cartridge may comprise a distal port. The cartridge may comprise a neck portion defining the distal port. The cartridge may further comprise a pierceable septum positioned over the distal port. The connector may be positioned along the tubular member.

In one embodiment, the assembly or system comprises an adhesive that provides for a permanent attachment of the connector to the reservoir.

In one embodiment, the connector comprises a coding feature provided along a portion of the body, in particular of the main body. The coding feature may cooperate with a corresponding coding feature provided by the drug delivery device. The corresponding coding feature may be provided by the reservoir holder, e.g. the cartridge holder, of the drug delivery device, for example. Alternatively or additionally, the corresponding coding feature may be provided by a dose setting member or a dose setting mechanism of the drug delivery device. The dose setting member or mechanism may be actuated to dispense a dose of drug from the reservoir retained in the reservoir holder.

The drug delivery device described herein may be a pen-type drug delivery device and/or an injection device. It may be a disposable or a reusable device.

According to an exemplary arrangement, a connector for use with a reservoir comprises a main body defining a bore extending from a proximal end to a distal end of the main body. The bore is configured for receiving the reservoir. An inner wall extends from the proximal end to the distal end and at least a portion of the inner wall is configured for attachment to an outer surface of a reservoir. An outer wall extends from the proximal end and the distal end. The outer wall is configured for attachment to a drug delivery device. The outer wall may be configured for attachment to a cartridge holder, a dose setting member, or a combination of the two.

In another arrangement, a cartridge for use with a drug delivery device comprises a tubular member. The tubular member comprises a bung located near a proximal end of the tubular member and a neck portion defining a distal port. A pierceable septum is positioned over the distal port. The pierceable septum may comprise a coding feature. A connector is positioned on the tubular member wherein the connector cooperates with a receiving member of a drug delivery device. The receiving member may be a cartridge holder, a dose setting member, or a combination of the two. In one arrangement, the connector is positioned near the proximal end of the tubular member. In the following text, a set of particularly advantageous aspects is specified. Within the set, numbers are used to identify specific aspects. This facilitates referencing the features of specific aspects in other aspects.

1. A connector for use with a reservoir, said connector comprising:

a main body defining a bore extending from a proximal end to a distal end of said main body, said bore configured for receiving said reservoir;

an inner wall extending from said proximal end to said distal end, at least a portion of said inner wall configured for attachment to an outer surface of a reservoir; and

an outer wall extending from said proximal end and said distal end, said outer wall configured for attachment to a drug delivery device.

2. The subject matter of aspect 1 wherein said connector comprises

a coding feature provided along a portion of said main body.

3. The subject matter of aspect 2 wherein said coding feature cooperates with a corresponding coding feature provided by said drug delivery device.

4. The subject matter of aspect 3 wherein said corresponding coding feature is provided by a cartridge holder of said drug delivery device.

5. The subject matter of aspect 3 wherein said corresponding coding feature is provided by a dose setting member of said drug delivery device.

6. The subject matter of aspect 1 wherein said connector comprises a flexible connector.

7. The subject matter of aspect 6 wherein said flexible connector comprises at least one flexible feature.

8. The subject matter of aspect 6 wherein said flexible connector comprises a plurality of crush ribs.

9. The subject matter of aspect 6 wherein said flexible connector comprises a multi-component flexible connector.

10. The subject matter of aspect 1 wherein said connector comprises a heat shrink material.

11. The subject matter of aspect 1 further comprising an adhesive that provides for a permanent attachment of said connector to said reservoir.

12. The subject matter of aspect 1 wherein said connector is positioned near a proximal end of said reservoir.

13. The subject matter of aspect 1 wherein said connector is positioned along an external surface of said reservoir by sliding said connector onto said proximal end of said reservoir.

14. The subject matter of aspect 1 wherein said connector comprises a snap fit connector such that said connector snaps together around a portion of said reservoir.

15. The subject matter of aspect 1 further comprising a heat shrink label positioned over a portion of said reservoir and said connector so that said heat shrink label maintains said connector on said reservoir.

16. The subject matter of aspect 15 wherein said heat shrink label terminates on a groove provided by said connector.

17. The subject matter of aspect 1 further comprising a retention feature provided on said main body of said connector

wherein said retention feature engages a groove provided along said proximal portion of said reservoir.

18. The subject matter of aspect 1 where said reservoir comprises a cartridge.

19. A cartridge for use with a drug delivery device, said cartridge comprising:

a tubular member, said tubular member comprising a bung located near a proximal end of said tubular member and a neck portion defining a distal port;

a pierceable septum positioned over said distal port; and

a connector positioned along said tubular member,

wherein said connector cooperates with a receiving member of a drug delivery device.

20. The subject matter of aspect 19 wherein said connector is configured to cooperate with a cartridge holder of said drug delivery device.

21. The subject matter of aspect 19 wherein said drug delivery device comprises a pen type drug delivery device.

22. A multi-component connector for use with a reservoir, said connector comprising:

an inner body configured for frictional attachment to an outer surface of said reservoir; and

an outer body having a connection mechanism for connecting to said inner body.

23. The subject matter of aspect 22 wherein said inner body comprises a plurality of inwardly directed radially fingers, said fingers providing said frictional engagement with said outer surface of said reservoir.

24. The subject matter of aspect 22 wherein said connection mechanism of said outer body comprises a thread, said thread used to connect to a thread provided on an external surface of said inner body.

25. The subject matter of aspect 22 wherein said connection mechanism of said outer body comprises a rib, said rib used to connect to a groove provided on an external surface of said inner body.

26. An assembly comprising a reservoir and a connector for use with the reservoir, said connector comprising a body configured for attachment to the reservoir, wherein the connector is adapted to be attached to a proximal portion of the reservoir and wherein a heat shrink label is positioned over a portion of said reservoir and said connector so that said heat shrink label maintains said connector on said reservoir, wherein said heat shrink label terminates on or in a groove provided by said connector.

Features described herein-above and below in conjunction with different aspects, embodiments, arrangements etc., may, of course, be combined with each other.

These as well as other features and advantages of various aspects of the present disclosure will become apparent to those of ordinary skill in the art by reading the following detailed description, with appropriate reference to the accompanying drawings.

### BRIEF DESCRIPTION OF THE DRAWINGS

Exemplary embodiments are described herein with reference to the drawings, in which:

FIG. 1 illustrates an exemplary pen type drug delivery device;

FIG. 2 illustrates a cartridge that may be loaded into a cartridge holder of the pen type drug delivery device illustrated in FIG. 1;

FIG. 3 illustrates a perspective view of the cartridge illustrated in FIG. 1 with a first arrangement of a connector;

FIG. 4 illustrates a close up view of a proximal end of the cartridge and connector arrangement illustrated in FIG. 3;

FIG. 5 illustrates a perspective view of the cartridge illustrated in FIG. 3 partially inserted into a cartridge holder that has been configured to receive the cartridge and connector combination;

FIG. 6 illustrates a perspective view of the cartridge illustrated in FIG. 3 and a dose setting member configured to receive the cartridge and connector combination;

FIG. 7 illustrates a top view of an alternative connector arrangement for use with the cartridge illustrated in FIG. 2;

FIG. 8 illustrates a perspective view of a proximal end of the cartridge illustrated in FIG. 1 with an alternative arrangement of a connector;

FIG. 9 illustrates a top view of yet another alternative connector arrangement for use with the cartridge illustrated in FIG. 2;

FIG. 10 illustrates a perspective view of yet another arrangement of a connector for use with the cartridge illustrated in FIG. 2;

FIG. 11 illustrates a close up perspective view of a proximal end of the connector arrangement illustrated in FIG. 10;

FIG. 12 illustrates a perspective view of a proximal end of the cartridge illustrated in FIG. 2 with another arrangement of a connector;

FIG. 13 illustrates a perspective view of a proximal end of the cartridge illustrated in FIG. 2 with a two part connector arrangement;

FIG. 14 illustrates a cross-section view of the two part connector arrangement illustrated in FIG. 13; and

FIG. 15 illustrates an alternative reservoir that may be used with the connector arrangements.

### DETAILED DESCRIPTION

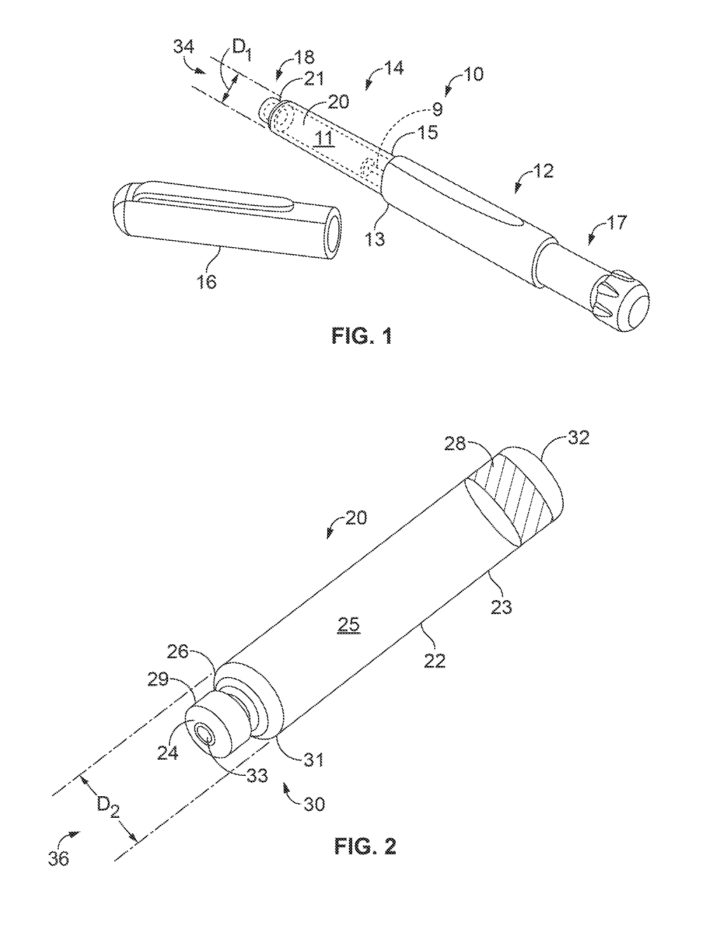

Referring to FIG. 1, there is shown a drug delivery device 10 in the form of a pen type drug delivery device. This drug delivery device 10 comprises a dose setting member or mechanism, a cartridge holder 14, and a removable cap 16. A proximal end 15 of the cartridge holder 14 and a distal end 13 of the dose setting mechanism 12 are secured together, preferably removably secured together. The dose setting mechanism 12 comprises a piston rod 9, such as a threaded piston rod that rotates when a dose is injected.

The term “drug” or “medicament”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(w-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-11e-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6—NH2,

H-Asn-(Glu)5-des Pro36, Pro37, Pro38 [Asp28] Exendin-4(1-39)-(Lys)6—NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6—NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6—NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

To inject a previously set dose, a double ended needle assembly is attached to a distal end 18 of the cartridge holder. Preferably, the distal end of the holder comprises a thread 21 (or other suitable connecting mechanism such as a snap lock, snap fit, form fit, or bayonet lock mechanism). In this manner, the needle assembly may be removably attached to the distal end of the holder. When the drug delivery device 10 is not in use, the removable cap 16 can be releasably retained over the cartridge holder 14.

An inner cartridge cavity 11 defined by the cartridge holder 14 is dimensioned and configured to securely receive and retain the cartridge 20 comprising a tubular barrel 22 having an outer surface 23. FIG. 2 illustrates a perspective view of the cartridge 20 that may be used with the drug delivery device illustrated in FIG. 1. Typically, a cartridge of this type is manufactured of glass and includes a generally tubular barrel 22 extending from a distal end 30 to a proximal end 32.

Referring now to FIG. 2, at the distal end 30, the cartridge 20 includes a smaller diameter neck 26 and this neck projects distally from the shoulder 31 of the barrel 22. Preferably, this smaller diameter neck 26 is provided with a large diameter annular bead and this bead extends circumferentially thereabout at the extreme distal end of the neck 26 and defines an opening. A pierceable seal or septum 33 is securely held across the opening defined by the neck and a metallic sleeve or ferrule 24. This ferrule 24, illustrated as having a smooth outer surface or sidewall 29, may be crimped around the circumferential bead at the distal end of the neck so as to hold the pierceable membrane in place.

The medicament 25 is pre-filled into the cartridge 20. This medicament 25 is retained within this cartridge, in part, by the pierceable seal 33, the ferrule 24, and the stopper 28. The stopper 28 is in sliding fluid-tight engagement with the inner tubular wall of the barrel 22. Axially directed forces acting upon the stopper 28 during dose injection or dose administration urges the medication 25 from the cartridge 20 though a double ended needle mounted onto the distal end 18 of the cartridge holder 14 and into the injection site. Such axial forces may be provided by the piston rod 9 working in unison with the dose setting member 12.

A portion of the cartridge holder 14 defining the cartridge holder cavity 11 is of substantially uniform diameter represented in FIG. 1 by D1 34. This diameter D1 34 is preferably slightly greater than the diameter D2 36 of the cartridge 20. The interior of the cartridge holder includes an inwardly-extending annular portion or stop that is dimensioned to prevent the cartridge 20 from moving within the cartridge holder 14. In this manner, when the cartridge 20 is loaded into the cavity 11 of the cartridge holder 14 and the cartridge holder 14 is then connected to the dose setting member 12, the cartridge 20 will be securely held within the cartridge cavity.

A number of doses of a medicament 25 may be dispensed from the cartridge 20. Preferably, the cartridge 20 contains a type of medicament that must be administered often, such as one or more times a day. One such medicament is insulin. Alternatively, the cartridge 20 contains a premix of such medicaments.

The dose setting mechanism 12 comprises a dose setter 17 at the proximal end of the dose setting mechanism. In one preferred arrangement, the dose setter 17 is rotated to set a dose. To administer this set dose, the user attaches the needle assembly comprising a double ended needle on the distal end of the cartridge holder. In this manner, the needle assembly pierces the seal 33 of the cartridge 20 and is therefore in liquid communication with the medicament 25. The user pushes on the dose setter 17 to inject the set dose. The same dose setting and dose administration procedure is followed until the medicament 25 in the cartridge is expended. Then, a new cartridge must be loaded in the device. To exchange an empty cartridge, the user is called upon to remove the cartridge holder 14 from the dose setting mechanism 12.

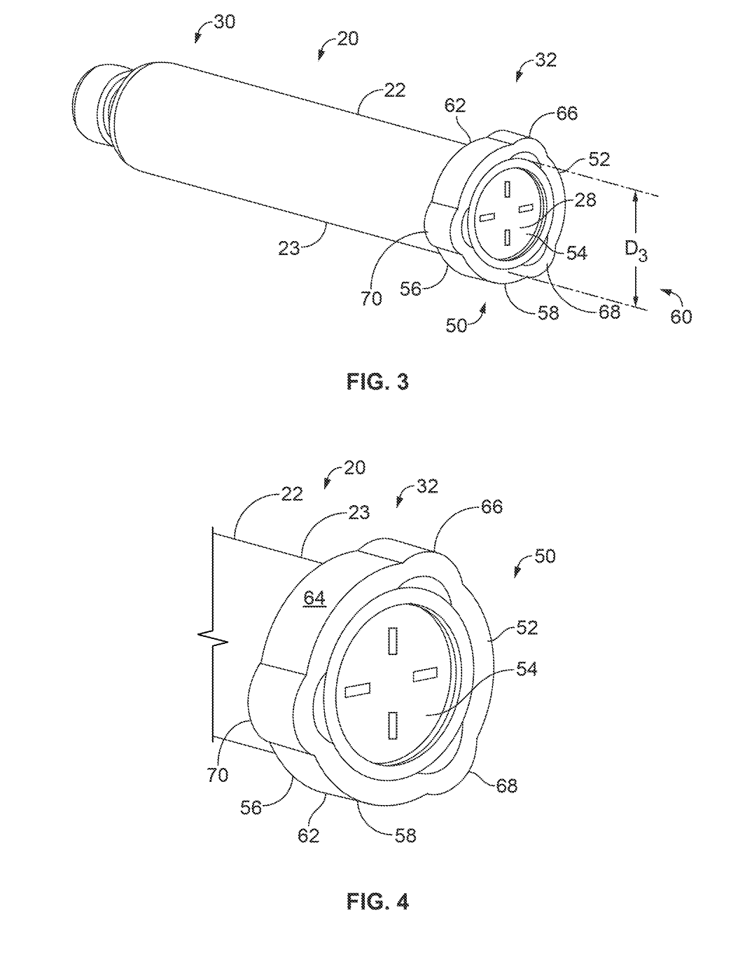

FIG. 3 illustrates a first arrangement of a cartridge connector 50 that may be mounted on or near a proximal end of a cartridge, such as the proximal end 32 of the cartridge 20 illustrated in FIG. 2. FIG. 4 illustrates a close up view of the proximal end 32 of the cartridge 20 and connector 50 illustrated in FIG. 3. Although the connector 50 is shown as being mounted near the proximal end of the cartridge, in an alternative arrangement, the connector could be mounted elsewhere along the outer surface of the cartridge, such as near the middle of the cartridge or near the distal end of the cartridge.

As illustrated, in this first arrangement, the cartridge connector 50 preferably comprises an integral or single piece connector. Cartridge connector 50 can be combined with a cartridge to form a cartridge arrangement that may be used with a drug delivery device, such as the pen type drug delivery device illustrated in FIG. 1. Most preferably, the connector 50 is customized to a particular cartridge holder so as to thereby prevent undesired cartridge cross use between cartridges having different connector configurations.

In this arrangement, in order to mount the connector 50 onto the cartridge 20, the connector is slid axially along the outer surface 23 of the cartridge so as to position the connector 50 near the proximal end 32 of the cartridge 20. To facilitate moving this connector in this fashion, the cartridge connector 50 comprises a material that is generally flexible in nature such as a polymer, for example polypropylene, acetal, or polyamide. Because it is flexible in diameter, the connector can flex over the outer surface of the cartridge but still remain in a rigid position.

Providing such a flexible cartridge connector offers a number of advantages. For example, the flexible connector 50 may be used with standard sized cartridges comprising glass cartridges, such as the glass cartridge 20 illustrated in FIG. 2. Because these standard type cartridges need not be modified in order to work with the proposed cartridge connector, the proposed connector can also reduce cost and technical risk. In addition, because of the flexible nature of the connector 50, the connector can overcome certain limitations that can naturally arise during the manufacturing process such as removing free play between the connector and the cartridge and/or the cartridge holder due to tolerance variations. Moreover, due to its flexible nature, such a connector may also eliminate the need for an adhesive to be used to provide additional security so as to make sure that the connector does not move along the outer surface of the cartridge. Application of such an adhesive may pose assembling and/or manufacturing concerns since it might be difficult to handle or control the application of such an adhesive either along the inner surface of the connector or along the outer surface of the cartridge in a high volume and/or high speed production or manufacturing environment.

Returning to FIGS. 3 and 4, the connector 50 comprises an essentially cylindrical shaped main body 52. This main body defines a centrally located bore 54. This bore 54 is geometrically configured for receiving the cartridge tubular barrel, preferably the proximal end 32 of this barrel. This bore 54 extends from a proximal end 58 to a distal end 56 of the main body 52 and, when a cartridge such as illustrated in FIG. 2 is inserted into this main body, the bore is positioned over a proximal portion of the cartridge. The connector 50 further comprises a vertically extending wall 62 extending from the proximal end of the main body 52 towards the distal end 56 of the main body 52.

Preferably, in this arrangement, the cartridge connector main body has a diameter D3 60 that is slightly smaller than an outer diameter of the cartridge 20 of the cartridge assembly illustrated as D2 36. Because this preferred main body 52 comprises a flexible material, this inner diameter may be stretched initially during assembly so that it can extend around the outer surface of the tubular barrel. FIGS. 3 and 4 show this flexible connector in a stressed state after it has been stretched over the proximal end of the cartridge 20. In this manner, the main body 52 is rigidly positioned onto the outer surface.

In this arrangement, the connector 50 comprises an outer surface. This outer surface is essentially a smooth outer surface. The outer surface comprises one or a plurality of features, such as flexible features and/or arc features. In the depicted embodiment, the outer surface comprises three flexible arc features 66, 68, 70. These features protrude radially outward. These features protrude away from the cartridge outer surface. Although only three flexible features are illustrated, a greater or a lesser number of such features may be including depending on, among other things, the main body material used, the dimensions of the main body, the shape of the cartridge, or the shape of the cooperating member of the drug delivery device or cartridge holder. As those of ordinary skill in the art will also recognize, the general geometrical shape or location of each such feature may be modified as well. One advantage of such arc features is that they can enhance the flexible nature of the main body 52. In addition, such features may also act as coding features so as to mechanically code this particular connector and cartridge arrangement to a specific drug delivery device.

Importantly, the connector main body 52 is dimensioned so as to cooperate with the drug delivery device, preferably either a cartridge holder or a dose setting member of the drug delivery device. More preferably, the main body 52 is dimensioned so as to cooperate with only a single drug delivery device to thereby prevent inadvertent cross use of cartridge assemblies and therefore inadvertent cross use of medicaments. As just one example, the outer surface of the main body may be dimensioned so as to cooperate with an inner recess or cavity of a cartridge holder of the drug delivery device, such as the inner cavity 11 of the cartridge holder 14 illustrated in FIG. 1.

Preferably, in one arrangement, the connector 50 may also comprise additional coding features. Such coding features could be applied in various positions and/or locations on the connector. As just one example, connector 50 may comprise one or more coding features in the form of a plurality of projections provided along a portion of the main body. These projections may be arranged so that they will cooperate with a cartridge holder that includes a cooperating coding mechanism in the form of recesses that allow the projections to sit within these recesses.

The connector 50 is intended for use with a drug delivery device, such as the device 10 illustrated in FIG. 1. More preferably, the connector 50 is intended for use with a cartridge holder similar to the cartridge holder 14 of FIG. 1. However, a preferred cartridge holder for use with the connector 50 would have a slightly modified inner cavity so as to accommodate the greater outer diameter of the connector 50, and in particular the outwardly radiating or outwardly directed flexible features 66, 68 and 70. For example, a proximal end of a modified cartridge holder would comprise receiving features that would be geometrically configured to receive these features 66, 68 and 70.

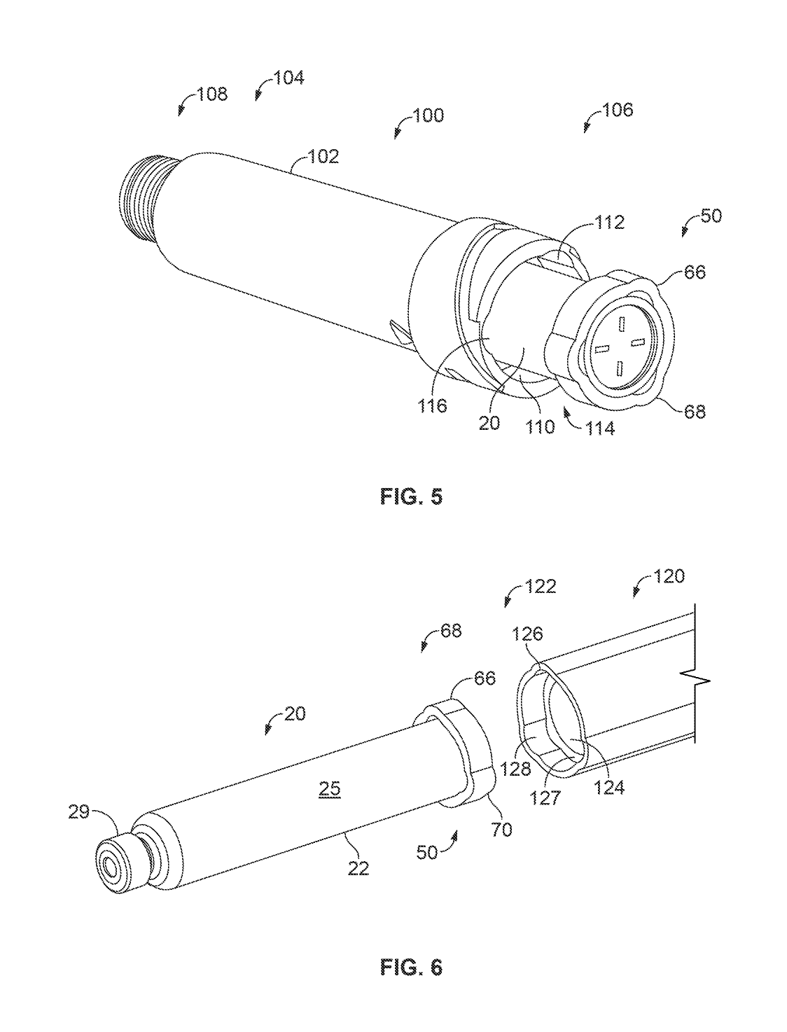

For example, FIG. 5 illustrates a perspective view of the cartridge illustrated in FIG. 3 partially inserted into a modified cartridge holder 100. As illustrated, the cartridge holder 100 comprises a generally tubular receiving body 102 extending from a distal end 104 to a proximal end 106. In one preferred arrangement, the distal end 104 comprises a thread connection 108 and is configured for receiving a double ended needle. Although a thread is illustrated, alternative releasable connection mechanisms may also be used.

The proximal end 106 of the cartridge holder 100 further comprises a connection mechanism so that that when combined, the cartridge holder, the cartridge and connector may be attached to a dose setting mechanism. As just one example, where the cartridge holder 100 is used for a reusable drug delivery device, this proximal end 106 may comprise a releasable coupling (e.g., a thread, a snap lock that is released through a thread, a bayonet connection or other similar form fit or snap fit connection). In this manner, the holder 100 may be releasably connected to the dose setting mechanism of a drug delivery device. Alternatively, where the cartridge holder 100 is used for a disposable drug delivery device, this proximal end 106 may comprise a non-releasable coupling (e.g., such as a non-releasable snap lock or snap-fit connection such as a groove and rib arrangement). In such a non-releasable arrangement, the holder 100 is permanently attached to the dose setting mechanism of a drug delivery device.

As illustrated, the cartridge holder 100 defines an internal cavity 110 and at the proximal end of the holder, this inner cavity 110 is mechanically configured to receive the cartridge and connector arrangement illustrated in FIG. 4. More preferably, at the proximal end 106 of the cartridge holder 100, there is provided an inner cavity having e.g. three grooves 112, 114, and 116 that are geometrically configured to mate with the three rounded protrusions 66, 68, 70 of the connector 50. In this manner, the cartridge 20 that is correctly coded with the connector 50 may be used with this cartridge holder 100. As such, a cartridge that is incorrectly coded with a different connector may not be able to be used with this cartridge holder 100.

In an alternative arrangement, rather than cooperate with the cartridge holder 100, the main body 52 of the connector 50 may be dimensioned so as to cooperate with the dose setting mechanism of a drug delivery device, such as the device illustrated in FIG. 1. That is, such a dose setting member may be modified so as to be configured to receive such a cartridge and connector arrangement as illustrated in FIG. 3. For example, as illustrated in FIG. 6, the dose setting mechanism 120 may comprise at a distal end 122 an inner cavity 124 having e.g. three grooves 126, 127, and 128 that are geometrically configured to mate with the three arc features or rounded protrusions 66, 68, 70 of the connector 50. In this manner, the cartridge 20 that is correctly coded with the connector 50 may be used with this dose setting mechanism 120. As such, a cartridge that is incorrectly coded with a different connector may not be able to be used with this dose setting mechanism 120.

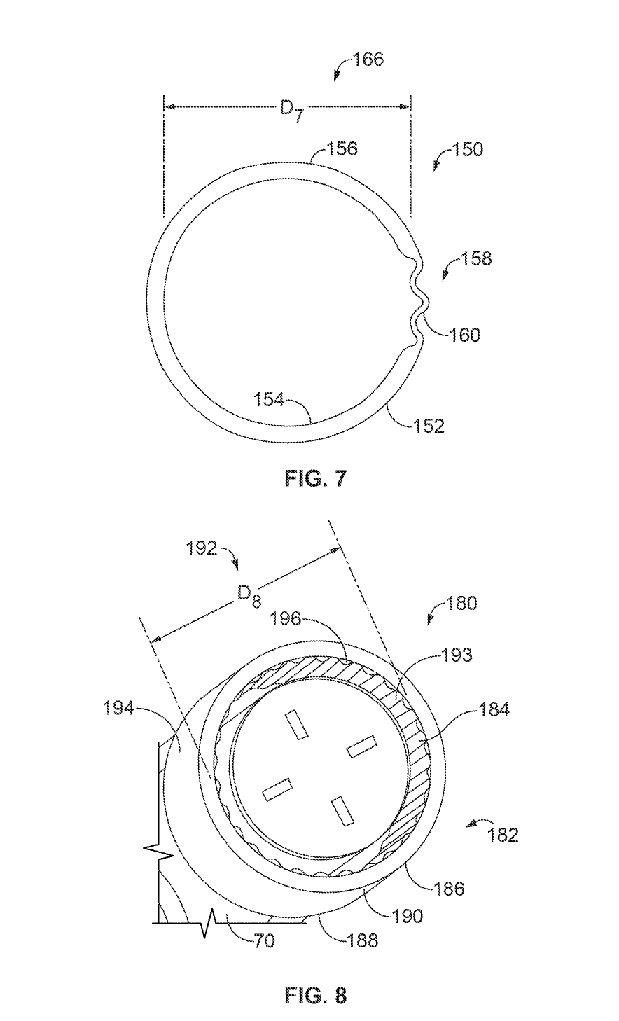

The connector 50 illustrated in FIG. 3 may be modified. For example, the connector 50 may be provided with a section of the connector that could deform as the connector is assembled to the cartridge. As just one example, the connector 50 may be provided with one or more thin-walled sections and/or undulations. For example, FIG. 7 illustrates such an alternative arrangement of the connector 50 illustrated in FIG. 3. In this connector arrangement 150 of FIG. 7, the connector has a similar configuration as connector 50 in that it comprises a unitary main body 152 and this main body comprises a generally smooth outer wall 156 and a generally smooth inner wall 154.

This main body also comprises a single thin-walled section 158 comprising an undulation 160. This single thin-walled section 158 allows a certain degree of flexibility of the main body 152 so that the main body can be stretched over an outer surface of a cartridge, such as the outer surface of the cartridge illustrated in FIG. 2. In this manner, an outer diameter D7 166 of the main body could be slightly smaller than the diameter of the cartridge outer surface. Although only one thin-wall section 158 or undulation 160 is illustrated, alternative arrangements comprising multiple thin-walled sections and/or undulations arrangements may also be used.

Similar to the connector illustrated in FIGS. 3 and 4, to use this connector arrangement 150 with a drug delivery device, such as a pen type drug delivery device, either or both the cartridge holder or the dose setting member could be modified so as to mechanically cooperate with the connector and cartridge arrangement. For example, a cartridge holder similar to the cartridge holder illustrated in FIG. 5 or similar to the modified dose setting member illustrated in FIG. 6 could be used but provided with a different geometrical inner housing configuration so as to cooperate with the outer surface or outer wall 156 and width of the connector 150.

In yet another alternative connector arrangement, a cartridge connector may be provided with a plurality of internally radiating or inwardly directed ribs, e.g. axial ribs. The ribs may be crush ribs. For example, FIG. 8 illustrates a connector arrangement 180 comprising a plurality of axial ribs. In such an arrangement, these ribs may be crushed during an assembly process. For example, they could be crushed as the connector is positioned along the outer surface of the cartridge.

As illustrated, the connector 180 is similar in structure as connector 50 illustrated in FIG. 1 in that it comprises an essentially cylindrical shaped main body 182. This main body 182 defines a centrally located bore 184. This bore 184 is geometrically configured for receiving a proximal end of a cartridge, such as the proximal end of the cartridge 30 illustrated in FIG. 2. This bore 184 extends from a proximal end 186 to a distal end 188 of the main body 182. When the cartridge 20 is inserted into this main body 182, the bore 184 is positioned over the proximal portion of the cartridge. The connector 180 further comprises a vertically extending wall 190 extending from the proximal end 186 of the main body 182 towards the distal end 188 of the main body 182.

Preferably, in this arrangement, the connector main body 182 has an inner diameter D8 192 that is slightly smaller than an outer diameter of the cartridge 2 of the cartridge illustrated as D2. FIG. 8 illustrates the connector 180 in a stretched state after assembly onto the outer surface of the cartridge. In this illustration, the cartridge is transparent, so that the connector 180 is illustrated as already being assembled onto a cartridge outer surface.

In this arrangement, the connector 180 comprises an inner surface 193 and an outer surface 194. As illustrated, the inner surface 193 comprises a plurality of internally radiating axial ribs 196. The outer surface 194 is essentially a smooth outer surface. Although no coding features are illustrated along this outer surface 194, such coding features could be provided (similar to the three flexible features 66, 68, and 70 illustrated in FIGS. 3 and 4). Naturally, a greater or a lesser number of such features may be including on the connector 180 depending on, among other things, the main body material used, the dimensions of the main body, the shape of the cartridge, the shape of the receiving cartridge holder, and/or the shape of the receiving dose setting mechanism.

With such a connector 180, a residual force in the connector may allow friction to retain the connector on the cartridge 20. To increase this retaining frictional force, the connector 180 may comprise a high friction material such as an elastomer, for example, rubber, ethylene propylene rubber (EPM), Thermoplastic Elastomer (TPE). For example, such a rubber component could comprise either a separate component part or alternatively be over-molded or co-molded onto the connector.

With this connector arrangement, an adhesive could also be used to retain the connector 180 on the cartridge 20. Alternatively, an adhesive could also serve the purpose of filling any potential gaps that might occur between the inner surface of the connector and the outer surface of the cartridge. Such gaps can often arise due to manufacturing tolerances and other production related circumstances.

The connector could alternatively be retained along the outer surface of the cartridge 20 by heat-shrinking it onto the cartridge outer surface. Again, this may occur either with or without an adhesive. Preferably, this heat shrink process occurs without heating the cartridge contents and therefore the connector could be placed on the cartridge before the cartridge is filled with its medicament 25.

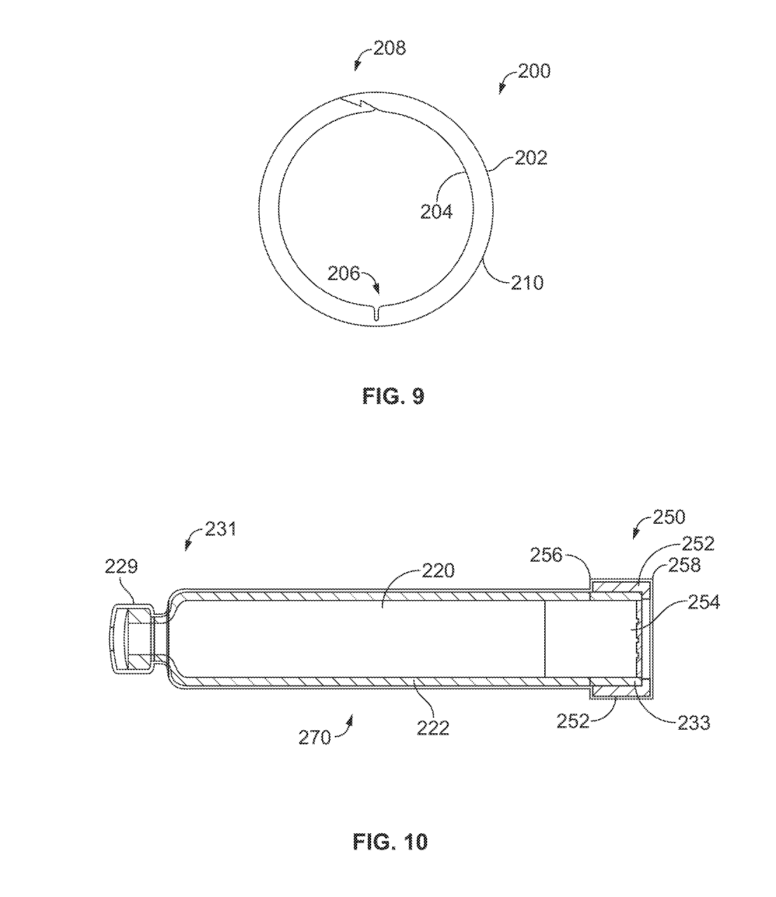

In one arrangement, the connector may snap together and either be one piece with a hinge or be in more than one piece. For example, FIG. 9 illustrates an alternative arrangement for a connector 200. Connector 200 comprises a hinged connector. Connector 200 comprises a generally tubular shape similar to the generally tubular shapes of the previously discussed connector arrangements 50, 150, and 180.

For example, the connector 200 comprises a circular main body 202 having an inner surface 204 and an outer surface 210. The main body defines a non-releasably hinge (see the region to which reference numeral 208 points) once both hinge portions are connected together. In addition, the connector 200 may be provided with a cut-out or recess 206 along the inner surface 204. This cut-out provides flexibility to the main body of the connector when the hinge is in the non-locked position prior to assembly.

To assemble this connector 200 with a cartridge, the connector could simply be snapped around the outer surface of the cartridge. One advantage of such a configuration is that it does not have to slide along the external cartridge surface or over any ridge at the open end of the cartridge.

FIG. 10 illustrates another arrangement of a cartridge connector 250 for use with a cartridge 220, similar to the cartridge 20 illustrated in FIG. 2. The cartridge comprises a tubular barrel 222. The distal end 231 of the cartridge 220 is covered by a septum 229 which seals the distal port of the cartridge. In FIG. 10, the connector is shown mounted on the most proximal end 233 of the cartridge 220, in particular on the tubular barrel 222. In this arrangement, connector 250 comprises a main body 252 defining a centrally located bore 254. This bore configured for receiving the most proximal end 233 of the cartridge 220. This bore 254 extends from a proximal end 258 along the main body 252 towards the distal end 256 of the connector 250. Preferably, this connector arrangement comprises a label 270 that is affixed over and around the connector and extends from the distal end 231 of the cartridge and over an open proximal end 233 of the cartridge to thereby surround the entire connector 250. In such an arrangement, the connector 250 and label 270 combination may act as a tamper-evident feature. In addition, the label 270 may be used to help retain the connector 250 to the cartridge 220. Alternatively, mechanical retention features such as a mechanical clip could be used to attach the connector 250 to the cartridge 220. Either or both the cartridge holder or dose setting mechanism would have to be mechanically configured to cooperate with the connector 250, similar to the cartridge holder and dose setting mechanism discussed with respect to FIGS. 5 and 6.

FIG. 11 illustrates another arrangement of a cartridge connector 300 for use with a cartridge 320 that may be used with a pen type drug delivery device, such as the drug delivery device illustrated in FIG. 1. Similar to the cartridge connector 250 illustrated in FIG. 10, this cartridge connector 300 comprises a cylindrically shaped main body 302 defining a centrally located bore 304. This bore 304 is geometrically configured for receiving a proximal end 333 of the cartridge 320 containing a medicament.

Similar to the connector and label arrangement illustrated in FIG. 10, this connector arrangement 300 also comprises a label 370. This label 370 that extends from a distal end of the cartridge 320 and towards the open proximal end 333 of the cartridge. Unlike the label arrangement 270 of FIG. 10 that completely surrounds the proximal end of the cartridge 333 along with the entire connector 250, in the label arrangement 370 of FIG. 11, the label extends along an outer surface of the cartridge 320 and terminates in a groove 374 provided on the main body of the connector 300. In such an arrangement, a distal portion of the label could wrap over the shoulder of the cartridge, as illustrated in the connector and label arrangement illustrated in FIG. 10.

In either connector arrangement illustrated in FIG. 10 or 11, the label may comprise a heat shrink label or alternatively an adhesive backed label. Preferably, the label may be predominantly clear. Such a clear label would allow a user to view the contents of the cartridge and perhaps any indicia provided by the cartridge provider that may be provided along the surface of the cartridge. Alternatively, the label could be colour-coded so as to provide indicia of cartridge contents, manufacturing information or the like.

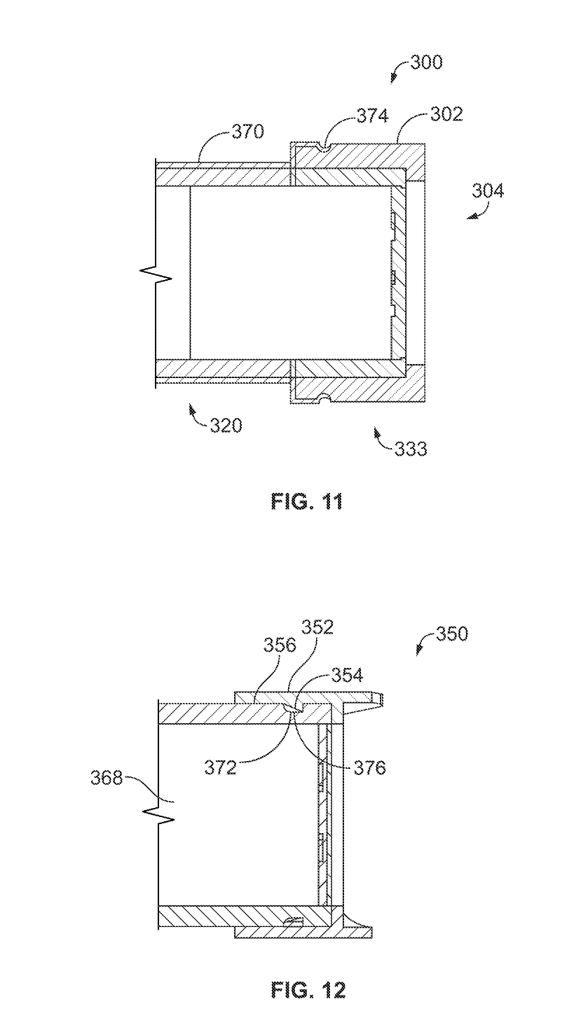

FIG. 12 illustrates a cross-section view of a proximal end of the cartridge illustrated in FIG. 2 of yet another arrangement of a connector 350. This alternative cartridge connector 350 is similar to the connector arrangements illustrated in FIGS. 10 and 11. That is, in this arrangement, the connector 350 comprises a smooth outer side wall 352 and also comprises a radial protrusion 354 provided along an inner side wall 356. In one alternative arrangement, cartridge connector 350 may comprise a coding feature in the form of a side wall 364 having a plurality of vertically offset surfaces (not explicitly shown). These surfaces are arranged so that they cooperate with a cartridge holder that includes a cooperating coding mechanism.

In this arrangement, the cartridge 368 comprises a retention feature 372 so as to retain the connector 350 in the illustrated position. For example, such a retention feature 372 could comprise a groove. In an alternative arrangement, instead of a groove, a ridge or radial protrusion on the cartridge could retain a groove provided along the inner wall of the connector. Alternatively, a flange could be added at the proximal end (i.e., the opened end), directed radially inwards or outwards. The extent of the retention feature may be any angle up to 360°, and may be intermittent around the circumference of the cartridge.

Either or both the cartridge holder or dose setting mechanism would have to be mechanically configured to cooperate with the connector 350, similar to the cartridge holder and dose setting mechanism discussed with respect to FIGS. 5 and 6.

With respect to the various cartridge connector arrangements discussed above, these arrangements are all illustrated as comprising a single piece connector. However, as those of skill in the art will recognize, such connector arrangements may comprise multi-components.

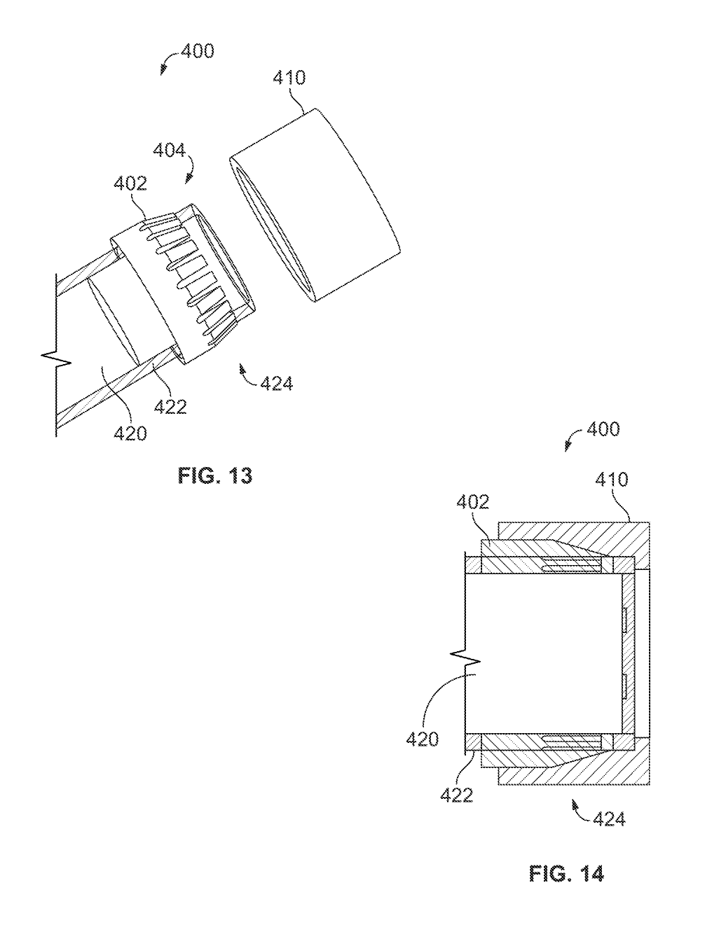

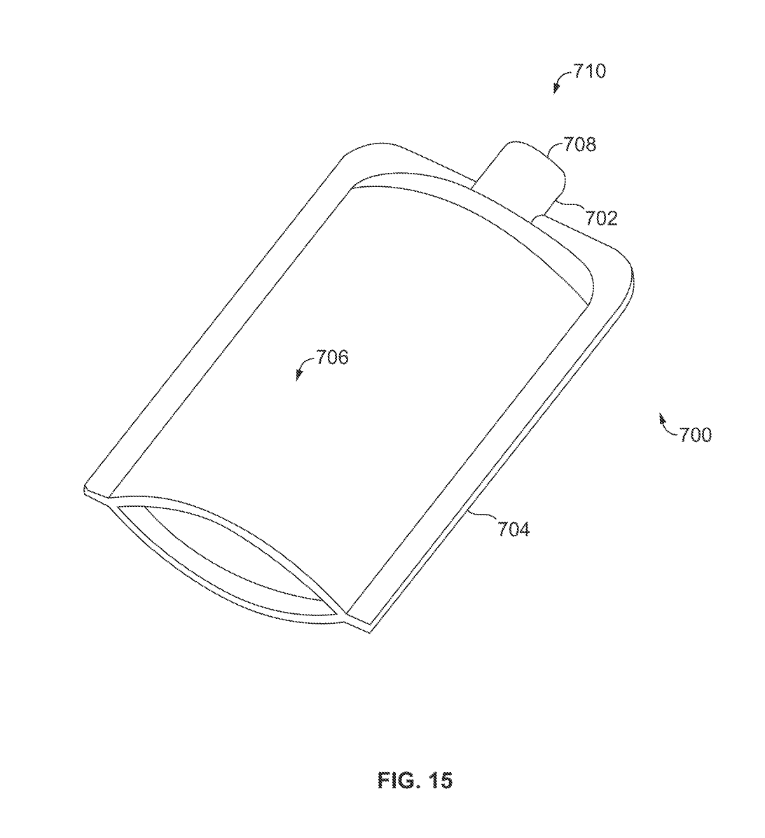

As just one example, FIG. 13 illustrates one arrangement of a multi-component connector 400 for use with a cartridge 420 that may be used with a drug delivery device, such as the drug delivery device illustrated in FIG. 1. FIG. 14 illustrates a perspective view of a proximal end 402 of a cartridge 420, similar to the cartridge 20 illustrated in FIG. 2 with a multi-component connector arrangement.

As illustrated, the connector 400 is retained along an outer surface 422 of the cartridge 420 near a proximal end 424 of the cartridge. As illustrated, this connector 400 is retained along this outer surface by means of compression. For example, in this arrangement, the connector 400 comprises an inner portion 402 and an outer portion 410 that are coupled together. These two parts 402, 410 may be releasably coupled by way of threads or the like. The inner portion 402 comprises a plurality of inwardly directed fingers 404 that are compressed inwards onto the cartridge outer surface by an angled face as the outer portion 410 is screwed onto the first connector portion 402. Together, the multi-component connector 400 is retained on the cartridge by friction.

A ratchet feature or an adhesive could be provided between the inner portion 402 and the outer portion 410 so as to ensure that the connector 400 does not become loose. Alternatively, the inner and ourter portions 402, 410 could combine to form a snap-fit coupling instead of a thread coupling. In yet another alternative arrangement, the plurality of flexible features 404 could be provided on the outer portion 410.

One advantage of this multi-component connector 400 is that is reduces free play due to various tolerances. In addition, certain multi-component connector embodiments can avoid the use of an adhesive, a manufacturing component that is often difficult to handle and control especially in a high volume manufacturing environment.

Although aimed primarily at the insulin market, the presently proposed cartridge connector may apply to other drugs. For example, the presently proposed application may apply to various types of medical and related devices, including the following examples:

An injector pen with a cartridge (e.g. 3 ml cylindrical glass cartridge) and a separate holder.

An injector pen with a cartridge (e.g. 3 ml cylindrical glass cartridge) non removably retained in a holder, so that the holder will be disposed of with the primary pack.

An injector pen where the primary pack attaches directly to the pen, e.g. an injection molded polymer cartridge.

Any drug delivery device, with any type of primary pack, e.g. inhaler, pouch.

In other applications, the proposed connector system may apply to any drug delivery device, with any type of reservoir or primary pack, e.g. inhaler, pouch. For example, FIG. 15 illustrates a drug reservoir 700 comprising a vessel 704 that contains a medicament 706. A stopper 708 is provided along a distal end of the vessel and is attached to the vessel so as to prevent the medicament 706 from exiting the vessel 704. A connector 702 is provided on the vessel near an output port 710 of the vessel. This output port 710 has a rigid neck and a connector 702 is provided along this neck.

The proposed cartridge connector system results in a number of advantages. For example, the proposed cartridge connector arrangements assist a user to distinguish between medicaments. This helps to ensure that a delivery device can only be used with a medicament for which the device is intended. Therefore, with the system applied to a cartridge to form a cartridge connector arrangement, this arrangement is prevented from being loaded into any other drug by loading a cartridge with an incorrect or unwanted connector. The proposed cartridge connector prevents a user from completing one or more of the following actions: fully inserting the cartridge assembly into an incorrect cartridge holder or attaching the cartridge and/or cartridge holder onto an incorrect dose setting mechanism.

The proposed cartridge connector arrangements also result in a low cost coding mechanism. For example, the proposed cartridge connectors do not require a large number of parts and can be manufactured in a cost effective manner. In one preferred arrangement, the proposed cartridge connector comprises a single, unitary part. In some arrangements, the cartridge connector comprises a single unitary component. In other arrangements, the cartridge connector comprises more than a single unitary component. Moreover, there are quite a large number of different connector configurations between the cartridge assembly and the cartridge holder that may be used. Consequently, with the various proposed connector schemes, a large number of medicaments can be distinguished from one another. In addition, with the proposed connector schemes, if a user attempts to load an incorrect cartridge into a cartridge holder and/or drug delivery device designed for a different cartridge and connector arrangement, the user will be alerted at an early stage of the assembly process.

With certain arrangements, the proposed system can be used to prevent errors during manufacturing. That is, during manufacturing, the system would be beneficial when inserting cartridge assemblies into disposable cartridge holders and/or disposable devices. With an incorrect drug (and hence incorrectly coded cartridge connector), the user is alerted at an early stage of assembly process.

Exemplary embodiments of the present disclosure have been described. Those skilled in the art will understand, however, that changes and modifications may be made to these arrangements without departing from the true scope and spirit of the present disclosure, which is defined by the claims.

1. An assembly comprising a reservoir and a connector for use with the reservoir said connector comprising a body configured for attachment to the reservoir, wherein the connector is adapted to be attached to a proximal portion of the reservoir and wherein a heat shrink label is positioned over a portion of said reservoir and said connector so that said heat shrink label maintains said connector on said reservoir.

2. The assembly of claim 1, wherein said heat shrink label terminates on or in a groove provided by said connector.

3. The assembly of claim 1 wherein the body comprises a main body defining a bore extending from a proximal end to a distal end of said main body, said bore configured for receiving said reservoir, an inner wall extending from said proximal end to said distal end, at least a portion of said inner wall configured for attachment to an outer surface of the reservoir, and an outer wall extending from said proximal end and said distal end, said outer wall configured for attachment to or cooperation with a drug delivery device.

4. The assembly of claim 1, wherein the connector is a multi-component connector, wherein the body comprises an inner body configured for frictional attachment to an outer surface of said reservoir, and wherein the body comprises an outer body having a connection mechanism for connecting to said inner body.

5. The assembly of claim 4, wherein said inner body comprises a plurality of inwardly directed radially fingers, said fingers being arranged to provide frictional engagement with said outer surface of said reservoir for the frictional attachment of the inner body to the outer surface.

6. The assembly of claim 4, wherein said connection mechanism of said outer body comprises a thread, said thread used to connect to a thread provided on an external surface of said inner body, or wherein said connection mechanism of said outer body comprises a rib, said rib used to connect to a groove provided on an external surface of said inner body.

7. The assembly of claim 1 wherein said connector comprises a flexible connector and wherein said flexible connector comprises: at least one flexible feature, and/or a multi-component flexible connector.

8. The assembly of claim 1, wherein said connector comprises a flexible connector and wherein said flexible connector comprises a plurality of crush ribs.

9. The assembly of claim 1, wherein said connector comprises a heat shrink material.

10. The assembly of claim 1, wherein said connector is positionable along an external surface of said reservoir by sliding said connector onto a proximal end of said reservoir.

11. The assembly of claim 1, wherein said connector comprises a snap fit connector such that said connector can snap together around a portion of said reservoir.

12. The assembly of claim 1, wherein the connector is attached to the reservoir and wherein the connector is configured to cooperate with or to attach to a receiving member of a drug delivery device.

13. The assembly of claim 1, wherein said connector is positioned at the proximal portion of said reservoir.

14. The assembly of claim 1, further comprising a retention feature provided on said body of said connector wherein said retention feature engages a groove provided along said proximal portion of said reservoir.

15. The assembly of claim 1, wherein said reservoir comprises a cartridge.

16. The assembly of claim 1, further comprising an adhesive that provides for a permanent attachment of said connector to said reservoir.

17. A drug delivery device comprising an assembly of claim 1, wherein the reservoir comprises a cartridge and the connector is attached to or cooperates with a cartridge holder of the drug delivery device.

18. The device of claim 17 wherein said connector comprises a coding feature provided along a portion of said body, said coding feature cooperating with a corresponding coding feature provided by said drug delivery device, wherein said corresponding coding feature is provided by the cartridge holder of said drug delivery device or wherein said corresponding coding feature is provided by a dose setting mechanism of said drug delivery device.


SEQUENCE LISTING (1 sequence)

SEQ ID NO: 1 - protein, 39 aa, Artificial Sequence
  Synthetic
SPPPAGSSPGGNKLWEIFLRVAEEEMQKSLDSTFTGEGH